(19) United States
(12) Patent Application Publication
(10) Pub. No.: US 2008/0214974 A1
(43) Pub. Date: Sep. 4, 2008

(54) Leg splint

(76) Inventor: Garry L. Roberts, Quinter, KS (US)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(21) Appl. No.: 11/713,304

(22) Filed: Mar. 1, 2007

(51) Int. Cl. A61F 5/00

(52) U.S. Cl. 602/23

(57) ABSTRACT

Methods and apparatus for strengthening and straightening livestock legs are provided. These methods and apparatus include a use of a splint that has a brace and a securing mechanism. The splint is secured to the forelimb of an animal.

### BACKGROUND OF THE INVENTION

1. Field

The invention relates generally to a device to strengthen livestock legs, and, more particularly, a device to correct weak front legs or straighten bent front legs in newborn livestock.

2. Background

Occasionally livestock, such as calves, are born too big for their front legs to support them. Calves and other livestock may also have front leg disorders or structural defects that make the legs weak and unable to support the animal.

Some veterinarians treat these leg weaknesses or disorders by casting the legs. This often leads to further complications and does not correct the problem. If the problem cannot be corrected, the animal never develops properly and becomes a financial loss for the farmer or rancher who owns the animal.

Thus, the need exists for a device to strengthen young livestock's legs.

### SUMMARY OF THE INVENTION

An object of this invention is to provide a device to help livestock's front legs develop.

A further object of this invention is to provide a device to strengthen livestock's front legs.

Another object of this invention is to provide a device to provide a device to correct structural problems in the front legs of livestock.

These and other objects of the present invention will become apparent to those skilled in the art upon reference to the following specification, drawings, and claims.

The present invention intends to overcome the difficulties encountered heretofore. To that end, a splint for the foreleg of an animal is provided. The splint includes a brace with a back support, foot base or plate and lip, and a means to secure the brace to the leg.

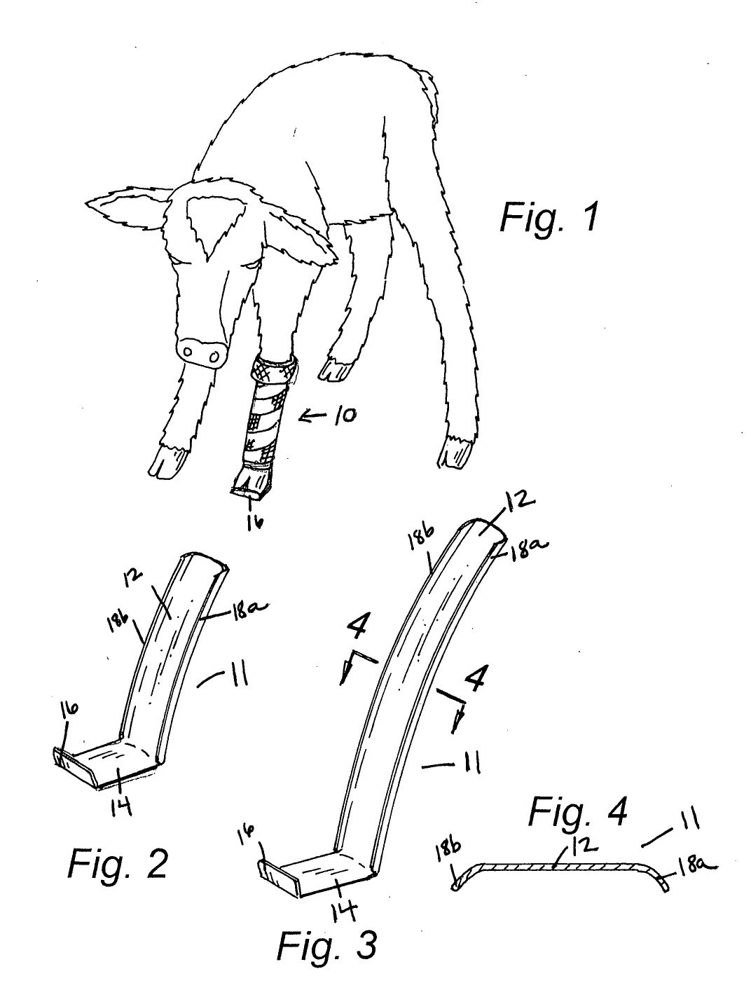

Brief Description of the Drawings FIG. 1 is a view of an animal wearing the splint of the present invention.

FIG. 2 is a view of one size of the splint of the present invention.

FIG. 3 is a view of a second size of the splint of the present invention.

FIG. 4 is a cross section of the brace of the present invention.

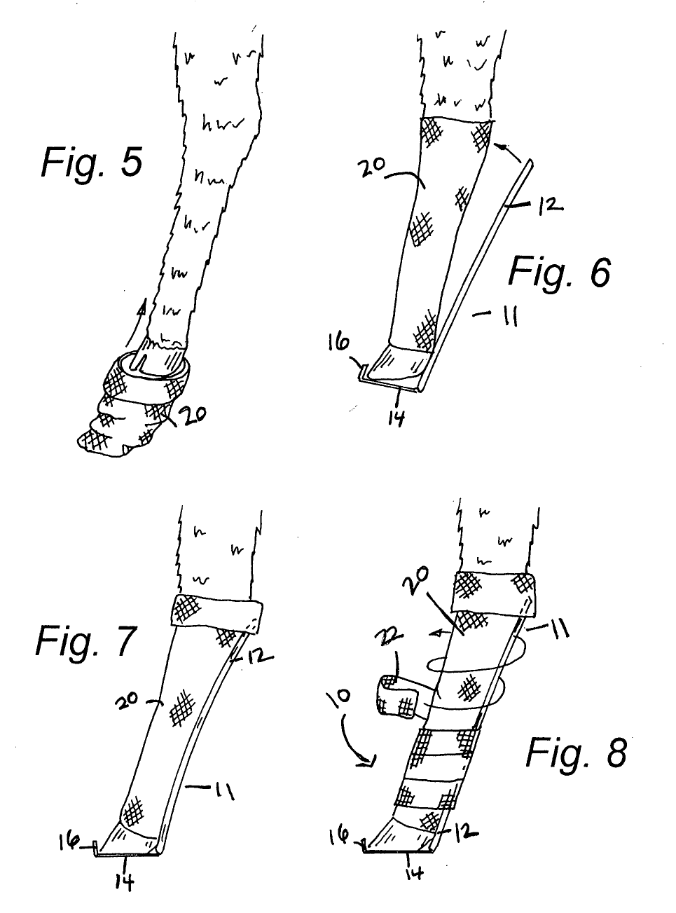

FIG. 5 is a view of the protective padding of the present invention.

FIG. 6 is a view of the placement of the brace of the present invention on an animal's leg.

FIG. 7 is a view of the brace of the present invention in place on an animal's leg.

FIG. 8 is a view of the binding of the brace of the present invention to an animal's leg.

### DETAILED DESCRIPTION OF THE INVENTION

Illustrated in FIG. 1 is a splint 10 for strengthening and straightening the front legs of livestock. The splint 10 corrects weak front legs. The splint 10 straightens and strengthens the front legs of newborn animals if the animal is too large at birth to be supported by its legs.

The splint 10 allows the posterior tendons of the ankle and lower leg to lengthen, while also allowing the anterior tendons and ligaments of the ankle and lower leg to shorten. Issues with the length of the tendons tend to arise due to the position of the calf or other animal in utero, and must be corrected once the animal is born. Without intervention, the animal will not develop properly and the farmer or rancher will experience a financial loss from that animal.

As shown in FIGS. 2-4, the splint 10 includes a brace 11. The brace 11 has a back 12, a plate 14, and a lip 16. The hoof of the animal stands on the plate 14 and the back 12 fits behind the leg of the animal. The lip 16 curves up around the front of the animal's hoof, as seen in FIGS. 1 and 6-8. The sides 18a, 18b of the back 12 are curved in its cross section to fit snugly against the animal's leg (FIG. 4).

FIG. 2 depicts a short brace 11 of the present invention. FIG. 3 shows a longer brace 11. The length of the brace 11 varies depending on whether the ankle alone is to be treated, or if both the leg and the ankle are to be treated. In general, the splint 10 is used to treat the lower forelimb of livestock. The length of the brace 11 used also varies with the size of the animal. The Figures illustrate the splint 10 used on calves, but the invention can be used on any type of livestock, including cows, horses, goats, and sheep.

The splint 10 may include protective padding 20. FIG. 5 illustrates the protective padding 20 that is placed on the animal's leg prior to placement of the brace 11. The protective padding 20 acts to protect the animal's leg from the brace 11. The padding 20 can be a fabric constraint, such as a tube sock and batting.

FIG. 6 shows the placement of the brace 11 on the animal's leg, over the protective padding 20. FIG. 7 illustrates the brace 11 in place over the protective padding 20. The splint 10 also includes a means to secure the brace 11 to the animal's leg. FIG. 8 illustrates one way to secure the brace 11 against the leg. A binding material 22, such as tape, straps, or bands secures the brace 11 to the leg. The binding or securing means 22 can be slid over the animal's foot and the brace 11, or the means 22 can be wrapped around the foot, ankle, leg and brace 11.

The brace 11 can be made from any hard or semi-hard material, such as metal or plastic.

Depending on the severity of the problem, the splint 10 is secured to the animal's leg for a period of weeks or months. In general, the length of treatment varies between one and three weeks.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention. For example, while this is intended for use with livestock, it could also be used for other types of animals, including zoo animals and companion animals.

1. A splint for the foreleg of an animal comprising:
a) a brace comprising a back member adapted to fit behind the foreleg of an animal and a foot base extended from the back member having a front end and a back end, wherein the front end of the foot base includes a lip; and
b) a means for attaching the brace to an animal's foreleg.

2. The splint of claim 1, further comprising padding placed between the brace and the animal's foreleg.

3. The splint of claim 1, wherein the back member is curved in cross-section.

4. A method of strengthening the foreleg of an animal comprising the steps of:
a) placing a brace behind the foreleg of an animal, wherein the brace comprises a back member, a foot base having a front end and a back end extended from the back member, and a lip at the front end of the foot base; and
b) securing the brace to the foreleg of the animal using a securing means until the foreleg is strengthened.

5. The method of claim 4, further comprising the step of placing padding between the animal's foreleg and the brace.

6. The method of claim 4, wherein the animal is a livestock animal.

7. The method of claim 4, wherein the animal is a newborn.

8. The method of claim 4, wherein the animal is selected from the group consisting of bovines, equines, porcines, sheep, and goats.

9. The method of claim 4, wherein the brace is secured to the foreleg for a period of 1 to 3 weeks.

10. The method of claim 4, wherein the brace is secured to the foreleg for a period of weeks.

11. The method of claim 4, wherein the brace is secured to the foreleg for a period of months.

12. A method of straightening the foreleg of an animal comprising the steps of:
a) placing a brace behind the foreleg of an animal, wherein the brace comprises a back member, a foot base having a front end and a back end extended from the back member, and a lip at the front end of the foot base; and
b) securing the brace to the foreleg of the animal using a securing means until the foreleg is straightened.
